(19) United States
(12) Patent Application Publication
(10) Pub. No.: US 2012/0214863 A1
(43) Pub. Date: Aug. 23, 2012

(54) Anti-miR-1 Therapy for Wound Healing

(75) Inventors: Chandan K. Sen, Upper Arlington, OH (US); Sashwati Roy, Upper Arlington, OH (US); Shani Goldberg-Shilo, Rehovot (IL)

(73) Assignee: THE OHIO STATE UNIVERSITY RESEARCH FOUNDATION, Columbus, OH (US)

(21) Appl. No.: 13/388,775

(22) PCT Filed: Aug. 4, 2010
(86) PCT No.: PCT/US10/44366

§ 371 Date: Apr. 11, 2012

Related U.S. Application Data
(60) Provisional application No. 61/231,506, filed on Aug. 5, 2009.

(51) Int. Cl. A61K 31/7088; C07H 21/00; C12N 15/113; A61P 17/02; A61K 31/7105; C12N 5/071; C12N 15/85; B82Y 5/00

(52) U.S. Cl. 514/44 A; 435/375; 435/366; 536/24.5; 435/455; 977/795; 977/906

(57) ABSTRACT

Methods for modulating gene expression in a skin cell by administering to the cell an amount of a therapeutic composition in an amount sufficient to modulate the expression of miR-1, and therapeutic compositions and uses thereof are disclosed.

### CROSS-REFERENCE TO RELATED APPLICATIONS

This application claims the benefit of U.S. Provisional Application No. 61/231,506 filed Aug. 5, 2009, the entire disclosure(s) of which is/are expressly incorporated herein by reference.

### STATEMENT REGARDING FEDERALLY SPONSORED RESEARCH

This invention was made with any Government support and the Government has rights in this invention under the NIH Grant No.GM069589, Project No. 747025.

### TECHNICAL FIELD AND INDUSTRIAL APPLICABILITY OF THE INVENTION

This invention is directed to methods for cutaneous wound healing which involve changes in the expression of specific miRNA at specific phases of wound healing, and therapeutic compositions therefor.

### BACKGROUND OF THE INVENTION

Repair of a defect in the human skin is a highly orchestrated physiological process involving numerous factors that act in a temporally resolved synergistic manner to re-establish barrier function by regenerating new skin. The inducible expression and repression of genes represents a key component of this regenerative process.

MicroRNAs (miRNAs or miRs) are endogenously expressed non-coding RNAs that regulate the expression of gene products by inhibition of translation and/or transcription in animals. miRNAs play a key role in skin morphogenesis and in regulating angiogenesis.

It is critically important to recognize that the understanding of cutaneous wound healing is incomplete without appreciating the functional significance of wound-induced miRNA. There is a critical need in the art for new strategies and compositions for the treatment of wounds generally, and chronic wounds specifically.

### SUMMARY OF THE INVENTION

The present invention is also based, in part, upon the discovery that the cutaneous wound healing process involves changes in the expression of specific miRNAs at specific phases of wound healing.

It is demonstrated herein that miR-directed strategies can be productive in wound healing.

Also described herein in a method where miR-directed therapies are used to target a whole cluster of genes regulated by the given miR. This method can be useful to orchestrate a multi-faceted healing response.

In a broad aspect, there is provided herein a method of modulating gene expression in a skin cell comprising: administering to the cell an amount of a therapeutic composition in an amount sufficient to modulate the expression of miR-1.

In certain embodiments, the expression of the miR-1 is down-regulated.

In certain embodiments, the therapeutic composition comprises an anti-miR-1 gene product.

In certain embodiments, the anti-miR-1 gene product is an isolated miR-1 nucleic acid.

In certain embodiments, the anti-miR-1 gene product is a recombinant nucleic acid.

In certain embodiments, the recombinant nucleic acid comprises an anti-miR-1 expression cassette.

In certain embodiments, the anti-miR-1 gene product is a synthetic nucleic acid.

In certain embodiments, the therapeutic composition comprises a double stranded nucleic acid molecule having one strand that is at least 95% complementary to at least a portion of a nucleic acid sequence encoding the anti-miR-1 gene product.

In certain embodiments, the anti-miR-1 gene product inactivates expression of miR-1.

In certain embodiments, the therapeutic composition can further include one or more cell-penetrating peptides. In certain embodiments, the cell penetrating peptide is linked to the anti-miR-1 gene product.

In certain embodiments, the anti-miR-1 gene product is administered topically to a wound.

In certain embodiments, the anti-miR-1 gene product is administered locally to a wound.

In certain embodiments, the anti-miR-1 gene product is included in a dressing that is applied topically to a wound.

In certain embodiments, the anti-miR-1 gene product is administered locally to a wound via injection.

In certain embodiments, the anti-miR-1 gene product is comprised in a pharmaceutical formulation. In certain embodiments, the pharmaceutical formulation is a lipid composition or a nanoparticle composition. In certain embodiments, the pharmaceutical formulation includes biocompatible and/or biodegradable molecules.

In another broad aspect, there is provided herein a method of treating a patient diagnosed with or suspected of having or suspected of developing a pathological skin condition or disease related to a gene modulated by miR-1, the method comprising: administering to the patient an amount of a therapeutic composition comprised of an anti-miR-1 gene product in an amount sufficient to modulate a cellular pathway or a physiologic pathway.

In certain embodiments, the cell is in a subject having, suspected of having, or at risk of developing, a skin disease or condition.

In certain embodiments, the condition associated with decreased vascularity wound healing disorders. In certain embodiments, the cell is a keratinocyte.

In another broad aspect, there is provided herein a method of treating or preventing a condition associated with decreased wound healing in a subject, the method comprising: administering to the subject a compound comprised of an anti-miR-1 gene product that regulates or enhances re-epithelialization, and/or wound healing in regular or compromised wounds, wherein the administering is sufficient to treat or prevent the condition in the subject.

In another broad aspect, there is provided herein a method of identifying a therapeutic composition that regulates or enhances re-epithelialization, and/or wound healing in regular or compromised wounds in vivo or in situ, the method comprising: i) contacting at least one cell or tissue with a test compound that regulates, or is believed to regulate the expression of miR-1; ii) measuring the bioavailability or biological activity of miR-1; and, iii) identifying a compound that regulates the bioavailability or biological activity of miR-1. In certain embodiments, the compound is a polypeptide, nucleic acid or small molecule.

In another broad aspect, there is provided herein a method of promoting healing of a wound, comprising: administering to at least one wound cell or tissue, an amount of an miR-1 inhibitory compound effective to promote healing of the wound.

In certain embodiments, the wound is comprised of cell or tissue that does not experience a decrease in expression of miR-1, which leads to pathogenesis and delayed would healing.

In certain embodiments, the wound is one or more of: a chronic wound; a diabetic ulcer; a pressure ulcer; an arterial ulcer; a venous ulcer; an acute wound; and/or a surgical wound.

In certain embodiments, the miR-1 inhibitory compound inhibits an activity of miR-1.

In certain embodiments, the miR-1 inhibitory compound inhibits transcription of a gene encoding miR-1.

In certain embodiments, the miR-1 inhibitory compound is administered locally to the wound.

In certain embodiments, the miR-1 inhibitory compound is included in a dressing that is applied topically to the wound.

In certain embodiments, the miR-1 inhibitory compound is administered locally to the wound via injection.

In another broad aspect, there is provided herein a composition useful for promoting healing of a wound comprising an amount of a miR-1 inhibitory compound effective to promote healing of wounded tissue.

In another broad aspect, there is provided herein a composition comprising an anti-miR-1 gene product, and a pharmaceutically acceptable carrier.

In another broad aspect, there is provided herein an article useful for dressing a wound, where at least one improvement includes an amount of a miR-1 inhibitory compound effective to promote healing of wounded tissue.

In another broad aspect, there is provided herein a vector comprising a nucleic acid sequence encoding at least one anti-miR-1 gene product in a manner that allows expression thereof.

In another broad aspect, there is provided herein a cell comprising the expression vector described herein. In certain embodiments, the cell is a mammalian cell. In certain embodiments, the mammalian cell is a human cell.

Other systems, methods, features, and advantages of the present invention will be or will become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1: miR-1 expression is down-regulated during cutaneous wound healing.

FIG. 2: Notch ligand delta expression is up-regulated during cutaneous wound healing.

FIG. 3A (control) and FIG. 3B (wound): Notch ligand delta is up-regulated during cutaneous wound healing during the re-epithelialization process of keratinocytes.

FIG. 4: miR-1 targets Notch ligand delta expression and regulates its expression in human Keratinocytes.

FIG. 5A (hours) and FIG. 5B (days): silencing miR-1 in human Keratinocytes induces an increase in cell proliferation.

FIG. 6A, FIG. 6B and FIG. 6: silencing miR-1 in human Keratinocytes induces an increase in cell migration.

FIG. 7: miR-1 down-regulation of expression is impaired in ischemic wounds.

### DETAILED DESCRIPTION OF THE PREFERRED EMBODIMENT

Throughout this disclosure, various publications, patents and published patent specifications are referenced by an identifying citation.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Definitions

The singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

It is contemplated that any embodiment discussed in this specification can be implemented with respect to any method, kit, reagent, or composition of the invention, and vice versa. Furthermore, compositions of the invention can be used to achieve methods of the invention.

As used herein, the term “microRNA species”, “microRNA”, “miRNA”, or “miR” refers to small, non-protein coding RNA molecules that are expressed in a diverse array of eukaryotes, including mammals. MicroRNA molecules typically have a length in the range of from 15 to 120 nucleotides, the size depending upon the specific microRNA species and the degree of intracellular processing. Mature, fully processed miRNAs are about 15 to 30, 15-25, or 20 to 30 nucleotides in length, and more often between about 16 to 24, 17 to 23, 18 to 22, 19 to 21, or 21 to 24 nucleotides in length. MicroRNAs include processed sequences as well as corresponding long primary transcripts (pri-miRNAs) and processed precursors (pre-miRNAs). Some microRNA molecules function in living cells to regulate gene expression via RNA interference. A representative set of microRNA species is described in the publicly available miRBase sequence database as described in Griffith-Jones et al., Nucleic Acids Research 32:D109-D111 (2004) and Griffith-Jones et al., Nucleic Acids Research 34:D 140-D144 (2006), accessible on the World Wide Web at the Wellcome Trust Sanger Institute website. MicroRNAs may also include synthetic RNA duplex and vector-encoded hairpin molecules, designed to mimic the miRNAs (Lim et al., 2005, Nature, 433:769773; Linsley et al., 2007, Mol. Cell. Biol., 27:2240-2252, which are incorporated by reference herein).

As used herein, the term “miR-specific inhibitor” refers to a nucleic acid molecule that is complementary, or essentially complementary to at least a portion of a microRNA molecule and inhibits its binding or activity towards its target gene transcripts. A miR-specific inhibitor may interact with the miRNA directly or may interact with the miRNA binding site in a target transcript, preventing its interaction with a miRNA. In some embodiments, the miR-specific inhibitor comprises a nucleotide sequence of at least 5 consecutive nucleotides, at least 6 consecutive nucleotides, at least 7 consecutive nucleotides, at least 8 consecutive nucleotides, or at least 9 nucleotides that are complementary to the seed region of a microRNA molecule (i.e. within positions 1 to 10 of the 5′ end of the microRNA molecule referred to as the “seed region”).

In a particular embodiment, the miR-specific inhibitor may comprise a nucleotide sequence of at least 6 consecutive nucleotides that are complementary to the seed region of a microRNA molecule. These consecutive nucleotides complementary to the microRNA seed region may also be referred to as microRNA binding sites. A miR-specific inhibitor may be a single stranded molecule. The miR-specific inhibitor may be chemically synthesized or may be encoded by a plasmid. In some embodiments, the miR-specific inhibitor comprises RNA. In other embodiments, the miR-specific inhibitor comprises DNA. In other embodiments, the miR-specific inhibitor may encompass chemically modified nucleotides and non-nucleotides. See, e.g. Brennecke et al., 2005, PLOS Biol. 3(3):pe85.

In some embodiments, a miR-specific inhibitor may be an anti-miRNA (anti-miR) oligonucleotide (see WO2005054494; Hutvagner et al., 2004, PLoS Biol. 2:E98; Orom et al., 2006, Gene 372:137-141;). Anti-miRs may be single stranded molecules. Anti-miRs may comprise RNA or DNA or have non-nucleotide components.

Alternative embodiments of anti-miRs may be as described above for miR-specific inhibitors. Anti-miRs anneal with and block mature microRNAs through extensive sequence complementarity. In some embodiments, an anti-miR may comprise a nucleotide sequence that is a perfect complement of the entire miRNA. In some embodiments, an anti-miR comprises a nucleotide sequence of at least 6 consecutive nucleotides that are complementary to the seed region of a microRNA molecule at positions 2-8 and has at least 50%, 60%, 70%, 80%, or 90% complementarity to the rest of the miRNA. In other embodiments, the anti-miR may comprise additional flanking sequence, complimentary to adjacent primary (pri-miRNA) sequences. Chemical modifications, such as 2′-O-methyl; LNA; and 2′-O-methyl, phosphorothioate, cholesterol (antagomir); 2′-O-methoxyethyl have been described for anti-miRs (WO2005054494; Hutvagner et al., 2004, PLoS Biol. 2:e98; Meister et al., 2004, RNA 10:544-50; Orom et al., 2006, Gene 372:137-41; WO2005079397; Krutzfeldt et al., 2005, Nature 438:685-689; Davis et al, 2006; Nucleic Acid Res. 34:2294-2304; Esau et al., 2006, Cell Metab. 3:87-98). Chemically modified anti-miRs are commercially available from a variety of sources, including but not limited to Sigma-Proligo, Ambion, Exiqon, and Dharmacon.

An “effective amount” or “therapeutically effective amount” of an anti-miR gene product or miR-specific inhibitor is an amount sufficient to produce the desired effect, e.g., inhibition of expression of a target sequence in comparison to the normal expression level detected in the absence of the miR-specific inhibitor. Inhibition of expression of a target gene or target sequence is achieved when the expression level of the target gene mRNA or protein is about 90%, 80%, 70%, 60%, 50%, 40%, 30%, 25%, 20%, 15%, 10%, 5%, or 0% relative to the expression level of the target gene mRNA or protein of a control sample. The desired effect of a miR-specific inhibitor may also be measured by detecting an increase in the expression of genes down-regulated by the miRNA targeted by the miR-specific inhibitor.

By “modulate” is meant that the expression of the gene, or level of RNA molecule or equivalent RNA molecules encoding one or more proteins or protein subunits, or activity of one or more proteins or protein subunits is up-regulated or down-regulated, such that expression, level, or activity is greater than or less than that observed in the absence of the modulator. For example, the term “modulate” can mean “inhibit,” but the use of the word “modulate” is not limited to this definition.

The term “gene expression”, as used herein, refers to the process of transcription and translation of a gene to produce a gene product, be it RNA or protein. Thus, modulation of gene expression may occur at any one or more of many levels, including transcription, post-transcriptional processing, translation, post-translational modification, and the like.

As used herein, the term “expression cassette” refers to a nucleic acid molecule, which comprises at least one nucleic acid sequence that is to be expressed, along with its transcription and translational control sequences. The expression cassette typically includes restriction sites engineered to be present at the 5′ and 3′ ends such that the cassette can be easily inserted, removed, or replaced in a gene delivery vector. Changing the cassette will cause the gene delivery vector into which it is incorporated to direct the expression of a different sequence.

As used herein, the term “phenotype” encompasses the meaning known to one of skill in the art, including modulation of the expression of one or more genes, as measured by gene expression analysis or protein expression analysis.

As used herein, the terms “wound” and “wound site” are generally defined as any location in the host that arises from traumatic tissue injury, or alternatively, from tissue damage either induced by, or resulting from, surgical procedures.

As used herein the term “nucleic acid” refers to multiple nucleotides (i.e., molecules comprising a sugar (e.g., ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term shall also include polynucleosides (i.e., a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art. Thus, the term nucleic acid also encompasses nucleic acids with substitutions or modifications, such as in the bases and/or sugars.

“MicroRNA flanking sequence” as used herein refers to nucleotide sequences including microRNA processing elements. “MicroRNA processing elements” are the minimal nucleic acid sequences which contribute to the production of mature microRNA from precursor microRNA. “Precursor miRNA,” termed “pri-miRNAs,” are processed in the nucleus into about 70 nucleotide pre-miRNAs, which fold into imperfect stem-loop structures.

The microRNA flanking sequences may be native microRNA flanking sequences or synthetic microRNA flanking sequences. A native microRNA flanking sequence is a nucleotide sequence that is ordinarily associated in naturally existing systems with microRNA sequences, i.e., these sequences are found within the genomic sequences surrounding the minimal microRNA hairpin in vivo. Synthetic microRNA flanking sequences are nucleotides sequences that are not found to be flanking to microRNA sequences in naturally existing systems. The synthetic microRNA flanking sequences may be flanking sequences found naturally in the context of other microRNA sequences. Alternatively, they may be composed of minimal microRNA processing elements which are found within naturally occurring flanking sequences and inserted into other random nucleic acid sequences that do not naturally occur as flanking sequences or only partially occur as natural flanking sequences.

The microRNA flanking sequences within the precursor microRNA molecule may flank one or both sides of the stem-loop structure encompassing the microRNA sequence. In certain embodiments, preferred structures have flanking sequences on both ends of the stem-loop structure. The flanking sequences may be directly adjacent to one or both ends of the stem-loop structure or may be connected to the stem-loop structure through a linker, additional nucleotides or other molecules.

As used herein a “stem-loop structure” refers to a nucleic acid having a secondary structure that includes a region of nucleotides which are known or predicted to form a double strand (stem portion) that is linked on one side by a region of predominantly single-stranded nucleotides (loop portion). The terms “hairpin” and “fold-back” structures are also used herein to refer to stem-loop structures. Such structures and terms are well known in the art. The secondary structure does not require exact base-pairing. Thus, the stem may include one or more base mismatches. Alternatively, the base-pairing may not include any mismatches.

As used herein, the terms “miR-1” and “miR-1 gene product” (which may be used interchangeably at times herein) generally refer to the nucleic acid encoding the miR-1 miRNA and homologues and variants thereof including conservative substitutions, additions, and deletions therein not adversely affecting the structure or function.

Preferably, miR-1 refers to the nucleic acid encoding miR-1, most preferably, miR-1 refers to the nucleic acid encoding a miR-1 family member from humans, and biologically active sequence variants of miR-1, including alleles, and in vitro generated derivatives of miR-1 that demonstrate miR-1 activity.

Sequence variants of miR-1 generally fall into one or more of three classes: substitutional, insertional or deletional variants. Insertions include 5′ and/or 3′ terminal fusions as well as intrasequence insertions of single or multiple residues. Insertions can also be introduced within the mature sequence of miR-1. These, however, ordinarily will be smaller insertions than those at the 5′ or 3′ terminus, on the order of 1 to 4 residues.

Insertional sequence variants of miR-1 are those in which one or more residues are introduced into a predetermined site in the target miR-1. Most commonly insertional variants are fusions of nucleic acids at the 5′ or 3′ terminus of miR-1.

Deletion variants are characterized by the removal of one or more residues from the miR-1 RNA sequence. These variants ordinarily are prepared by site specific mutagenesis of nucleotides in the DNA encoding miR-1, thereby producing DNA encoding the variant, and thereafter expressing the DNA in recombinant cell culture. However, variant miR-1 fragments may be conveniently prepared by in vitro synthesis. The variants typically exhibit the same qualitative biological activity as the naturally-occurring analogue, although variants also are selected in order to modify the characteristics of miR-1.

Substitutional variants are those in which at least one residue sequence has been removed and a different residue inserted in its place. While the site for introducing a sequence variation is predetermined, the mutation per se need not be predetermined. For example, in order to optimize the performance of a mutation at a given site, random mutagenesis may be conducted at the target region and the expressed miR-1 variants screened for the optimal combination of desired activity. Techniques for making substitution mutations at predetermined sites in DNA having a known sequence are well known.

Nucleotide substitutions are typically of single residues; insertions usually will be on the order of about from 1 to 10 residues; and deletions will range about from 1 to 30 residues. Deletions or insertions preferably are made in adjacent pairs; i.e., a deletion of 2 residues or insertion of 2 residues. Substitutions, deletion, insertions or any combination thereof may be combined to arrive at a final construct. Changes may be made to increase the activity of the miRNA, to increase its biological stability or half-life. All such modifications to the nucleotide sequences encoding such miRNA are encompassed.

A DNA isolate is understood to mean chemically synthesized DNA, cDNA or genomic DNA with or without the 3′ and/or 5′ flanking regions. DNA encoding miR-1 can be obtained from other sources by: a) obtaining a cDNA library from cells containing mRNA; b) conducting hybridization analysis with labeled DNA encoding miR-1 or fragments thereof (usually, greater than 100 bp) in order to detect clones in the cDNA library containing homologous sequences; and, c) analyzing the clones by restriction enzyme analysis and nucleic acid sequencing to identify full-length clones.

As generally used herein, nucleic acids and/or nucleic acid sequences are homologous when they are derived, naturally or synthetically, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity is routinely used to establish homology. Higher levels of sequence similarity, e.g., 30%, 40%, 50%, 60%, 70%, 80%, 90%, 95% or 99% or more can also be used to establish homology. Methods for determining sequence similarity percentages (e.g., BLASTN using default parameters) are generally available. Software for performing BLAST analyses is publicly available through the National Center for Biotechnology Information.

An “antisense” oligonucleotide or polynucleotide is a nucleotide sequence that is substantially complementary to a target polynucleotide or a portion thereof and has the ability to specifically hybridize to the target polynucleotide.

Embodiments of the invention concern nucleic acids that perform the activities of or inhibit endogenous miRNAs when introduced into cells. In certain aspects, nucleic acids are synthetic or non-synthetic miRNA. Sequence-specific miRNA inhibitors can be used to inhibit sequentially or in combination the activities of one or more endogenous miRNAs in cells, as well those genes and associated pathways modulated by the endogenous miRNA.

The present invention concerns, in some embodiments, short nucleic acid molecules that function as miRNAs or as inhibitors (anti-miRs) of miRNA in a cell. The term “short” refers to a length of a single polynucleotide that is 15, 16, 17, 18, 19, 20, 21, 22, 23, 24, 25, 50, 100, or 150 nucleotides or fewer, including all integers or ranges derivable there between. The nucleic acid molecules are typically synthetic.

The term “synthetic” refers to a nucleic acid molecule that is isolated and not produced naturally in a cell. In certain aspects the sequence (the entire sequence) and/or chemical structure deviates from a naturally-occurring nucleic acid molecule, such as an endogenous precursor miRNA or miRNA molecule or complement thereof. While in some embodiments, nucleic acids of the invention do not have an entire sequence that is identical or complementary to a sequence of a naturally-occurring nucleic acid, such molecules may encompass all or part of a naturally-occurring sequence or a complement thereof. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA or an inhibitor thereof.

The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together. In certain aspects, synthetic miRNA of the invention are RNA or RNA analogs. miRNA inhibitors may be DNA or RNA, or analogs thereof. miRNA and miRNA inhibitors of the invention are collectively referred to as “synthetic nucleic acids.”

In certain embodiments, synthetic miRNA have (a) a “miRNA region” whose sequence or binding region from 5′ to 3′ is identical or complementary to all or a segment of a mature miRNA sequence, and (b) a “complementary region” whose sequence from 5′ to 3′ is between 60% and 100% complementary to the miRNA sequence in (a). In certain embodiments, these synthetic miRNA are also isolated, as defined above. The term “miRNA region” refers to a region on the synthetic miRNA that is at least 75, 80, 85, 90, 95, or 100% identical, including all integers there between, to the entire sequence of a mature, naturally occurring miRNA sequence or a complement thereof. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA or complement thereof.

The term “complementary region” or “complement” refers to a region of a nucleic acid or mimetic that is or is at least 60% complementary to the mature, naturally occurring miRNA sequence. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there may be a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In other embodiments of the invention, there are synthetic nucleic acids that are miRNA inhibitors. A miRNA inhibitor is between about 17 to 25 nucleotides in length and comprises a 5′ to 3′ sequence that is at least 90% complementary to the 5′ to 3′ sequence of a mature miRNA. In certain embodiments, a miRNA inhibitor molecule is 17, 18, 19, 20, 21, 22, 23, 24, or 25 nucleotides in length, or any range derivable therein. Moreover, an miRNA inhibitor may have a sequence (from 5′ to 3′) that is or is at least 70, 75, 80, 85, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature miRNA, particularly a mature, naturally occurring miRNA. One of skill in the art could use a portion of the miRNA sequence that is complementary to the sequence of a mature miRNA as the sequence for a miRNA inhibitor. Moreover, that portion of the nucleic acid sequence can be altered so that it is still comprises the appropriate percentage of complementarily to the sequence of a mature miRNA.

In some embodiments, of the invention, a synthetic miRNA or inhibitor contains one or more design element(s). These design elements include, but are not limited to: (i) a replacement group for the phosphate or hydroxyl of the nucleotide at the 5′ terminus of the complementary region; (ii) one or more sugar modifications in the first or last 1 to 6 residues of the complementary region; or, (iii) noncomplementarity between one or more nucleotides in the last 1 to 5 residues at the 3′ end of the complementary region and the corresponding nucleotides of the miRNA region. A variety of design modifications are known in the art, see below.

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an aminohexyl phosphate group, an acetyl group, 2′O-Me (2′oxygen-methyl), DMTO (4,4′-dimethoxytrityl with oxygen), fluorescein, a thiol, or acridine, though other replacement groups are well known to those of skill in the art and can be used as well. This design element can also be used with a miRNA inhibitor.

Additional embodiments concern a synthetic miRNA having one or more sugar modifications in the first or last 1 to 6 residues of the complementary region (referred to as the “sugar replacement design”). In certain cases, there can be one or more sugar modifications in the first 1, 2, 3, 4, 5, 6 or more residues of the complementary region, or any range derivable therein. In additional cases, there can be one or more sugar modifications in the last 1, 2, 3, 4, 5, 6 or more residues of the complementary region, or any range derivable therein. It will be understood that the terms “first” and “last” are with respect to the order of residues from the 5′ end to the 3′ end of the region. In particular embodiments, the sugar modification is a 2′O-Me modification, a 2′F modification, a 2′H modification, a 2′amino modification, a 4′thioribose modification or a phosphorothioate modification on the carboxyl group linked to the carbon at position 6′. In further embodiments, there are one or more sugar modifications in the first or last 2 to 4 residues of the complementary region or the first or last 4 to 6 residues of the complementary region. This design element can also be used with a miRNA inhibitor. Thus, a miRNA inhibitor can have this design element and/or a replacement group on the nucleotide at the 5′ terminus, as discussed above.

In other embodiments of the invention, there is a synthetic miRNA or inhibitor in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region.

It is contemplated that synthetic miRNA of the invention have one or more of the replacement, sugar modification, or noncomplementarity designs. In certain cases, synthetic RNA molecules have two of them, while in others these molecules have all three designs in place.

The miRNA region and the complementary region may be on the same or separate polynucleotides. In cases in which they are contained on (or in) the same polynucleotide, the miRNA molecule will be considered a single polynucleotide. In embodiments in which the different regions are on separate polynucleotides, the synthetic miRNA will be considered to be comprised of two polynucleotides.

When the RNA molecule is a single polynucleotide, there can be a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein. In certain embodiments, the linker is between 3 and 30 residues (inclusive) in length.

In addition to having a miRNA or inhibitor region and a complementary region, there may be flanking sequences as well at either the 5′ or 3′ end of the region. In some embodiments, there is or is at least 1, 2, 3, 4, 5, 6, 7, 8, 9, 10 nucleotides or more, or any range derivable therein, flanking one or both sides of these regions.

“Sample” refers to a sample of cells, or tissue or fluid isolated from an organism or organisms, including but not limited to, for example, skin, plasma, serum, spinal fluid, lymph fluid, synovial fluid, urine, tears, blood cells, organs, tumors, and also to samples of in vitro cell culture constituents (including but not limited to conditioned medium resulting from the growth of cells in cell culture medium, recombinant cells and cell components).

As used herein, “subject”, as refers to an organism or to a cell sample, tissue sample or organ sample derived therefrom, including, for example, cultured cell lines, biopsy, blood sample, or fluid sample containing a cell. For example, an organism may be an animal, including but not limited to, a cow, a pig, a mouse, a rat, a chicken, a cat, a dog, etc., and is usually a mammal, such as a human.

Vectors

In one embodiment, the nucleic acid encoding a miRNA gene product (e.g., anti-miR) is on a vector. These vectors include a sequence encoding a mature anti-miR gene product and in vivo expression elements. In certain embodiments, these vectors include a sequence encoding a pre- or pri-miRNA and in vivo inhibition elements such that expression of the pre- or pri-miRNA is inhibited and prevented from being processed in vivo into a mature miRNA.

Vectors include, but are not limited to, plasmids, cosmids, phagemids, viruses, other vehicles derived from viral or bacterial sources that have been manipulated by the insertion or incorporation of the nucleic acid sequences for producing the microRNA, and free nucleic acid fragments which can be attached to these nucleic acid sequences. Viral and retroviral vectors are a preferred type of vector and include, but are not limited to, nucleic acid sequences from the following viruses: retroviruses, such as: Moloney murine leukemia virus; Murine stem cell virus, Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes viruses; vaccinia viruses; polio viruses; and RNA viruses such as any retrovirus. One of skill in the art can readily employ other vectors known in the art.

Viral vectors are generally based on non-cytopathic eukaryotic viruses in which non-essential genes have been replaced with the nucleic acid sequence of interest. Non-cytopathic viruses include retroviruses, the life cycle of which involves reverse transcription of genomic viral RNA into DNA with subsequent proviral integration into host cellular DNA. Retroviruses have been approved for human gene therapy trials. Genetically altered retroviral expression vectors have general utility for the high-efficiency transduction of nucleic acids in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., N.Y. (1990) and Murry, E. J. Ed. “Methods in Molecular Biology,” vol. 7, Humana Press, Inc., Cliffton, N.J. (1991).

The “in vivo inhibitor elements” are any regulatory nucleotide sequence, such as an inhibitor sequence, which inhibits or prevents the expression of the nucleic acid from producing the microRNA. In some embodiments, the miR-specific inhibitor may be an anti-miR, antagomir, and target mimics. In a particular embodiment, the miR-specific inhibitor comprises a polynucleic acid molecule comprising a nucleotide sequence of at least six contiguous nucleotides that is complementary miR-1.

Gene Products

By “gene product” is meant any product of transcription or translation of the genes, whether produced by natural or artificial means. The term “gene product” is intended to include the mRNA or protein encoded by a gene, or cDNA that corresponds to the encoded mRNA. As used herein, an “isolated” gene product is one which is altered or removed from the natural state through human intervention. For example, an RNA naturally present in a living animal is not “isolated.” A synthetic RNA, or an RNA partially or completely separated from the coexisting materials of its natural state, is “isolated.” An isolated RNA can exist in substantially purified form, or can exist in a cell into which the RNA has been delivered. Thus, a miR-1 gene product which is deliberately delivered to or expressed in a cell, such as a keratinocyte cell, is considered an “isolated” gene product.

As used herein, a “miR-1 mediated cell” is cell isolated from a subject suffering from a miR-1 mediated disorder. A miR-1 mediated cell can be identified by detecting an increase or presence of miR-1 gene products in the cell, or by detecting a phenotype in the cell.

The miR-1 gene products can be obtained using a number of standard techniques. For example, the gene products can be chemically synthesized or recombinantly produced using methods known in the art. Preferably, the RNA products are chemically synthesized using appropriately protected ribonucleoside phosphoramidites and a conventional DNA/RNA synthesizer. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

The miR-1 gene products can be administered to a subject by any means suitable for delivering the gene products to cells of the subject. For example, the miR-1 gene products can be administered by methods suitable to transfect cells of the subject with miR-1 gene products, or with nucleic acids comprising sequences encoding the miR-1 gene products. The cells can be transfected directly with the miR-1 gene products (as these are nucleic acids), or can be transfected with nucleic acids comprising sequences encoding the miR-1 gene products. Preferably, the cells are transfected with a plasmid or viral vector comprising sequences encoding the miR-1 gene products.

Therapeutic Methods

The present invention may also be used to stimulate the growth and repair of skin tissue. In wounds which involve injury to areas of the skin, and particularly in the case of massive burns, it is important that the skin grow very rapidly in order to prevent infections, reduce fluid loss, and reduce the area of potential scarring. Skin damage resulting from burns, punctures, cuts and/or abrasions may be treated using the gene activated matrices of the present invention. Skin disorders such as psoriasis, atopic dermatitis or skin damage arising from fungal, bacterial and viral infections or treatment of skin disorders such as melanoma, may also be treated using the methods of the invention.

In some embodiments of this invention, modulation of small non-coding RNA levels, expression or function is achieved via oligomeric compounds which target a further RNA associated with the particular small non-coding RNA. This association can be a physical association between that RNA and the particular small non-coding RNA such as, but not limited to, in an RNA or ribonucleoprotein complex. This association can also be within the context of a biological pathway, such as but not limited to, the regulation of levels, expression or function of a protein-encoding mRNA or its precursor by a small non-coding RNA.

As such, the invention provides for modulation of the levels, expression or function of a target nucleic acid where the target nucleic acid is a messenger RNA whose expression levels and/or function are associated with one or more small non-coding RNAs. The messenger RNA function or processing may be disrupted by degradation through an antisense mechanism, including but not limited to, RNA interference, or RNase H, as well as other mechanisms wherein double stranded nucleic acid structures are recognized and degraded, cleaved, sterically occluded, sequestered or otherwise rendered inoperable.

Compounds and Compositions

The compounds or compositions of the present invention may also interfere with the function of endogenous RNA molecules. The functions of RNA to be interfered with can include, for example, nuclear events such as replication or transcription as the compounds of the present invention could target or mimic small non-coding RNAs in these cellular processes. Replication and transcription, for example, can be from an endogenous cellular template, a vector, a plasmid construct or otherwise. The functions of RNA to be interfered with can include cytoplasmic events such as translocation of the RNA to a site of protein translation, translocation of the RNA to sites within the cell which are distant from the site of RNA synthesis, translation of protein from the RNA, splicing of the RNA to yield one or more RNA species, RNA signaling and regulatory activities, and catalytic activity or complex formation involving the RNA which may be engaged in or facilitated by the RNA as the compounds of the present invention could target or mimic small non-coding RNAs in these cellular processes.

As used herein, the term “modulation” and “modulation of expression” refers to either an increase (stimulation) or a decrease (inhibition) in the amount or levels of a small non-coding RNA, nucleic acid target, an RNA or protein associated with a small non-coding RNA, or a downstream target of the small non-coding RNA (e.g., a mRNA representing a protein-coding nucleic acid that is regulated by a small non-coding RNA). Inhibition is a suitable form of modulation and small non-coding RNA is a suitable target nucleic acid.

As used herein, the term “modulation of function” refers to an alteration in the function of the small non-coding RNA or an alteration in the function of any cellular component with which the small non-coding RNA has an association or downstream effect.

The specificity and sensitivity of compounds and compositions can also be harnessed by those of skill in the art for therapeutic uses. Antisense oligomeric compounds have been employed as therapeutic moieties in the treatment of disease states in animals, including humans. Antisense oligonucleotide drugs, including ribozymes, have been safely and effectively administered to humans and numerous clinical trials are presently underway. It is thus established that oligomeric compounds can be useful therapeutic modalities that can be configured to be useful in treatment regimes for the treatment of cells, tissues and animals, especially humans.

For therapeutics, an animal, preferably a human, suspected of having a disease or disorder presenting conditions that can be treated, ameliorated, or improved by modulating the expression of a selected small non-coding target nucleic acid is treated by administering the compounds and compositions.

For example, in one non-limiting embodiment, the methods comprise the step of administering to or contacting the animal, an effective amount of a modulator or mimic to treat, ameliorate or improve the conditions associated with the disease or disorder. The compounds of the present invention effectively modulate the activity or function of the small non-coding RNA target or inhibit the expression or levels of the small non-coding RNA target.

In one embodiment, the activity or expression of the target in an animal is inhibited by about 10%. In another embodiment the activity or expression of a target in an animal is inhibited by about 30%. Further, the activity or expression of a target in an animal is inhibited by 50% or more, by 60% or more, by 70% or more, by 80% or more, by 90% or more, or by 95% or more. In another embodiment, the present invention provides for the use of a compound of the invention in the manufacture of a medicament for the treatment of any and all conditions disclosed herein.

The reduction of target levels may be measured in serum, adipose tissue, liver or any other body fluid, tissue or organ of the animal known to contain the small non-coding RNA or its precursor. Further, the cells contained within the fluids, tissues or organs being analyzed contain a nucleic acid molecule of a downstream target regulated or modulated by the small non-coding RNA target itself.

The oligomeric compounds and compositions of the invention can be utilized in pharmaceutical compositions by adding an effective amount of the compound or composition to a suitable pharmaceutically acceptable diluent or carrier. Use of the oligomeric compounds and methods of the invention may also be useful prophylactically.

The oligomeric compounds and compositions of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption.

The oligomeric compounds and compositions of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the oligomeric compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligomeric compounds of the invention can be prepared. In addition, larger oligomeric compounds that are processed to supply, as cleavage products, compounds capable of modulating the function or expression of small non-coding RNAs or their downstream targets are also considered prodrugs.

The term “pharmaceutically acceptable salts” refers to physiologically and pharmaceutically acceptable salts of the compounds and compositions of the invention: i.e., salts that retain the desired biological activity of the parent compound and do not impart undesired toxicological effects thereto. Suitable examples include, but are not limited to, sodium and postassium salts.

The present invention also includes pharmaceutical compositions and formulations that include the oligomeric compounds and compositions of the invention. The pharmaceutical compositions of the present invention may be administered in a number of ways depending upon whether local or systemic treatment is desired and upon the area to be treated.

Administration may be topical, epidermal and transdermal, oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated bandagesand the like may also be useful.

Oligomeric compounds may be formulated for delivery in vivo in an acceptable dosage form, e.g. as parenteral or non-parenteral formulations. Parenteral formulations include intravenous (IV), subcutaneous (SC), intraperitoneal (IP), intravitreal and intramuscular (IM) formulations, as well as formulations for delivery via pulmonary inhalation, intranasal administration, topical administration, etc.

In some embodiments, the subject may be a human. In certain embodiments, the subject may be a human patient. In certain embodiments, the subject may be in need of modulation of expression of one or more genes as discussed in more detail herein. In some particular embodiments, the subject may be in need of inhibition of expression of one or more genes as discussed in more detail herein. In particular embodiments, the subject may be in need of modulation, i.e. inhibition or enhancement, of a nucleic acid target in order to obtain therapeutic indications discussed in more detail herein.

In some embodiments, non-parenteral (e.g. oral) oligomeric compound formulations according to the present invention result in enhanced bioavailability of the compound.

As used herein, the term “bioavailability” refers to a measurement of that portion of an administered drug which reaches the circulatory system (e.g. blood, especially blood plasma) when a particular mode of administration is used to deliver the drug. Enhanced bioavailability refers to a particular mode of administration's ability to deliver oligonucleotide to the peripheral blood plasma of a subject relative to another mode of administration. For example, when a non-parenteral mode of administration (e.g. an oral mode) is used to introduce the drug into a subject, the bioavailability for that mode of administration may be compared to a different mode of administration, e.g., an IV mode of administration.

In general, an oral composition's bioavailability is said to be “enhanced” when its relative bioavailability is greater than the bioavailability of a composition substantially consisting of pure oligonucleotide, i.e. oligonucleotide in the absence of a penetration enhancer.

Tissue bioavailability refers to the concentration of compound in a tissue. Tissue bioavailability may be measured in test subjects by a number of means. Tissue bioavailability may be modified, e.g. enhanced, by one or more modifications to the oligomeric compound, by use of one or more carrier compounds or excipients. In general, an increase in bioavailability will result in an increase in tissue bioavailability.

Topical oligomeric compound compositions according to the present invention may comprise one or more “ penetration enhancers,” also known as “absorption enhancers” or simply as “penetration enhancers.” Accordingly, some embodiments of the invention comprise at least one oligomeric compound in combination with at least one penetration enhancer. In general, a penetration enhancer is a substance that facilitates the transport of a drug across cell membrane(s) associated with the desired mode of administration. Accordingly, it is desirable to select one or more penetration enhancers that facilitate the uptake of one or more oligomeric compounds, without interfering with the activity of the compounds, and in such a manner the compounds can be introduced into the body of an animal without unacceptable side-effects such as toxicity, irritation or allergic response.

Embodiments of the present invention provide compositions comprising one or more pharmaceutically acceptable penetration enhancers, and methods of using such compositions, which result in the improved bioavailability of oligomeric compounds administered via non-parenteral modes of administration. In some embodiments, compositions for non-parenteral administration include one or more modifications from naturally-occurring oligonucleotides (i.e. full-phosphodiester deoxyribosyl or full-phosphodiester ribosyl oligonucleotides). Such modifications may increase binding affinity, nuclease stability, cell or tissue permeability, tissue distribution, or other biological or pharmacokinetic property. Modifications may be made to the base, the linker, or the sugar. In some embodiments, compositions for administration to a subject, and in particular oral compositions for administration to an animal or human subject, can comprise modified oligonucleotides having one or more modifications for enhancing affinity, stability, tissue distribution, or other biological property.

Oral compositions for administration of non-parenteral oligomeric compounds and compositions of the present invention may be formulated in various dosage forms such as, but not limited to, tablets, capsules, liquid syrups, soft gels, suppositories, and enemas. The term “alimentary delivery” encompasses e.g. oral, rectal, endoscopic and sublingual/buccal administration. A common requirement for these modes of administration is absorption over some portion or all of the alimentary tract and a need for efficient mucosal penetration of the nucleic acid(s) so administered.

As used herein, the term “pharmaceutical carrier” or “excipient” refers to a pharmaceutically acceptable solvent, suspending agent or any other pharmacologically inert vehicle for delivering one or more oligomeric compounds to an animal. The excipient may be liquid or solid and is selected, with the planned manner of administration in mind, so as to provide for the desired bulk, consistency, etc., when combined with an oligomeric compound and the other components of a given pharmaceutical composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, EXPLOTAB); and wetting agents (e.g., sodium lauryl sulphate, etc.).

The pharmaceutical, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

One of skill in the art will recognize that formulations are routinely designed according to their intended use, i.e. route of administration.

Formulations for topical administration include those in which the oligomeric compounds of the invention are in admixture with a topical delivery agent such as lipids, liposomes, fatty acids, fatty acid esters, steroids, chelating agents and surfactants. Lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligomeric compounds and compositions of the invention may be encapsulated within liposomes or may form complexes thereto, in particular to cationic liposomes. Alternatively, they may be complexed to lipids, in particular to cationic lipids.

Certain embodiments of the invention provide pharmaceutical compositions containing one or more of the compounds and compositions of the invention and one or more other therapeutic agents that function by a non-antisense mechanism.

When used with the oligomeric compounds of the invention, such therapeutic agents may be used individually, sequentially, or in combination with one or more other such therapeutic agents. Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of oligomeric compounds and compositions of the invention and other drugs are also within the scope of this invention. Two or more combined compounds such as two oligomeric compounds or one oligomeric compound combined with further compounds may be used together or sequentially.

In another embodiment, compositions of the invention may contain one or more of the compounds and compositions of the invention targeted to a first nucleic acid target and one or more additional oligomeric compounds targeted to a second nucleic acid target. Alternatively, compositions of the invention may contain two or more oligomeric compounds and compositions targeted to different regions, segments or sites of the same target. Two or more combined compounds may be used together or sequentially.

Dosage

As used herein, an “effective amount” of miR-1 gene products is an amount sufficient to inhibit proliferation of a miR-1 mediated disorder cell in a subject suffering from a miR-1 mediated disorder.

One skilled in the art can readily determine an effective amount of the miR-1 gene products to be administered to a given subject, by taking into account factors such as the size and weight of the subject; the extent of disease penetration; the age, health and sex of the subject; the route of administration; and whether the administration is regional or systemic.

An effective amount of the miR-1 gene products can also be based on the approximate or estimated body weight of a subject to be treated. Preferably, such effective amounts are administered parenterally or enterally. For example, an effective amount of the miR-1 gene products administered to a subject can range from about 5-3000 micrograms/kg of body weight, and is preferably between about 700-1000 micrograms/kg of body weight, and is more preferably greater than about 1000 micrograms/kg of body weight. One skilled in the art can also readily determine an appropriate dosage regimen for the administration of the miR-1 gene products to a given subject. For example, the miR-1 gene products can be administered to the subject once (e.g., as a single injection or deposition). Alternatively, the gene products can be administered once or twice daily to a subject for a period of from about three to about twenty-eight days, more preferably from about seven to about ten days. In a preferred dosage regimen, the miR-1 gene products are administered once a day for seven days. Where a dosage regimen comprises multiple administrations, it is understood that the effective amount of the miR-1 gene products administered to the subject can comprise the total amount of gene product administered over the entire dosage regimen.

The formulation of therapeutic compounds and compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligomeric compounds, and can generally be estimated based on EC₅₀s found to be effective in in vitro and in vivo animal models. In general, dosage is from 0.01 μg to 100 g per kg of body weight, from 0.1 μg to 10 g per kg of body weight, from 1.0 μg to 1 g per kg of body weight, from 10.0 μg to 100 mg per kg of body weight, from 100 μg to 10 mg per kg of body weight, or from 1 mg to 5 mg per kg of body weight, and may be given once or more daily, weekly, monthly or yearly, or even once every 2 to 20 years. Persons of ordinary skill in the art can easily determine repetition rates for dosing based on measured residence times and concentrations of the drug in bodily fluids or tissues.

Following successful treatment, it may be desirable to have the patient undergo maintenance therapy to prevent the recurrence of the disease state, wherein the oligomeric compound is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight, once or more daily, to once every 20 years.

Methods of Treatment

In a particular aspect, there is described herein a method for inhibiting the expression of miR-1 in the cells of an organism comprising administering to the organisms an inhibitorily effective amount of one or more anti-miR-1 gene products that inhibit miR-1 expression in the cells of the organism. In certain embodiments, the anti-miR-1 gene product is administered to an organism afflicted with a wound healing disorder.

In another aspect, there is provided herein a therapeutic composition for administration to a patient in need of therapy for wound healing, comprising an isolated nucleic acid for the expression in the cells of the patient of an effective amount of an anti-miR-1 gene product to inhibit expression of miR-1.

In another aspect, there is provided herein a method for increasing the expression of Notch ligand delta in the cells of an organism comprising administering to the organism an effective amount of one or more antagonistic miRNAs that bind to one or more endogenous miRNAs and reverse the inhibition of Notch ligand delta.

In another aspect, there is provided herein a method for modulating expression of miR-1 in wound cells, the method comprising contacting an wound cell with an agent, which agent either reduces the functional level of at least miR-1.

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference.

The following examples are intended to illustrate certain preferred embodiments of the invention and should not be interpreted to limit the scope of the invention as defined in the claims, unless so specified.

### EXAMPLES

The present invention is also based, in part, upon the discovery that miR-1 expression levels are down-regulated during cutaneous wound healing. This down-regulation is impaired in non-healing wounds.

The present invention is also based, in part, upon the discovery that down-regulation of miR-1 induces an increase in keratinocytes migration and proliferation. miR-1 drives the re-epithelialization process.

A proven target of miR-1, Notch ligand delta, is up-regulated in migrating and proliferating epidermis. In addition, inhibiting miR-1 in human keratinocytes induces an increase in Notch ligand delta expression.

miR-1 expression in non-healing ischemic wounds is not down-regulated compared to normal wounds. Treating chronic or acute wounds with anti-miR-1 will enhance re-epithelialization, and therefore, enhance wound healing in regular or compromised wounds.

Methods:

In vivo: 8×16 mm wounds were made on the backs of 8 weeks old B6 mice. The skin excised was saved as control. Wound edge was collected at days 2, 7, 14 post wounding and frozen in liquid nitrogen and then kept in −80° C. Control skin and wound edge were grinded and then homogenized to extract miRNA using nirvana miRNA isolation kit (AMBION). Skin and wound edge were lysed with a protein lyses buffer using a tissue lyser machine.

miRNA were quantitated and Taqman® assays were run to detect the expression level of miR-1. snoRNA 202 was used as a house keeping small RNA.

Target protein expression-Notch ligand delta and house keeping-GAPDH were assayed using a western blot.

In addition: on the backs of 8 weeks old B6 mice, 6 mm punch wounds were applied. Wounds were collected at day 7 and freshly frozen in OCT. Cryosections were made, and immunohistochemistry was performed to detect Notch ligand delta expression and localization in normal and wounded skin. Zeiss microscope and Axiovision software were used.

Third, on the backs of 8 weeks old B6 mice, an ischemic flap was performed and 3 mm punch wounds were made to result in a non-ischemic and an ischemic wound on the back of the same animal. At day 7, wounds were excised and same miRNA isolation and detection was performed as mentioned earlier.

In vitro: human keratinocyte cell line (HaCaT) was used. Cells were seeded in 12 well plates 0.15xE6/1 ml/well in antibiotic free media. The next day, the cells were transfected with anti-miR-1 reagent and a non-targeting control, using transfection reagent Dharmafect-1 (all from Dharmafect). After 72 h, cells collected and used for protein extraction, or reseeded for assays.

Protein expression: 70 μg of protein was used to detect the expression of Notch ligand delta and GAPDH.

Cell proliferation: 10,000 cells were seeded in 96 well plate and assayed every 24 h for cell proliferation using CY Quant assay.

Cell migration: 0.2xE6/500 μl media cells were seeded in 4 well plates. The next day a scratch was made with a 10 μl tip in the middle of the wells. Cell migration and closure of the scratch was imaged every 2 h using a Zeiss microscope and axiovision software at 10× magnification.

Results

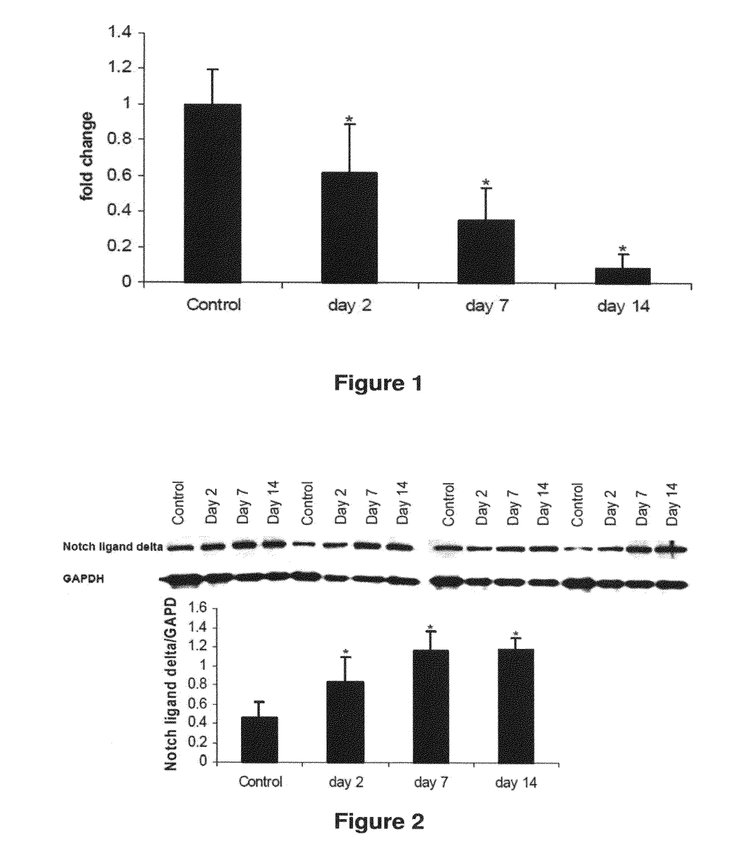

FIG. 1 shows that miR-1 expression is down-regulated during cutaneous wound healing. 8×16 mm wounds were created on the back skin of B6 mice. Wound edges were collected at days 2, 7, 14. Skin cut for the wound was saved as control. miRNA were isolated, and Taqman® real-time assay for the detection of miR-1 was performed. snoRNA202 was used as the housekeeping gene. The experiments were paired. Each wound had its own control N=7 paired, p<0.05 compared to control.

FIG. 2 shows that the Notch ligand delta expression is up-regulated during cutaneous wound healing. 8×16 mm wounds were created on the back skin of B6 mice. Wound edges were collected at days 2, 7, 14. Skin cut for the wound was saved as control. Protein was extracted and Western blot was run. 50 μg of protein was loaded. N=4 paired, p<0.05 compared to control.

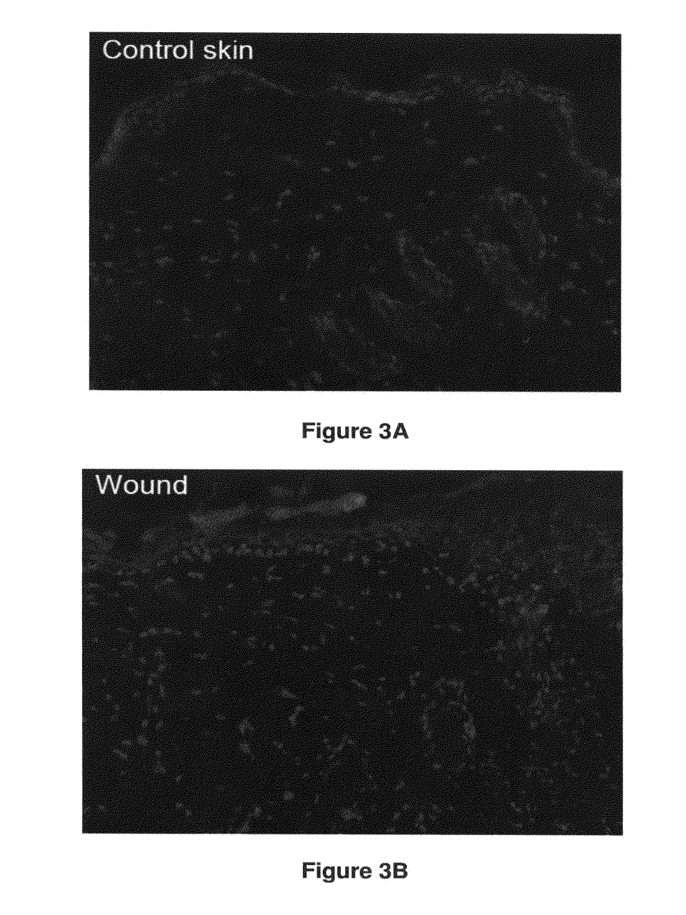

FIG. 3A (control) and FIG. 3B (wound) show that the Notch ligand delta is up-regulated during cutaneous wound healing during the re-epithelialization process of keratinocytes. 6 mm punch wounds were created on the back skin of B6 mice. Wound samples were collected at day 7 with surrounding intact skin that served as control, and freshly frozen in OCT. An immunohistochemistry was performed.

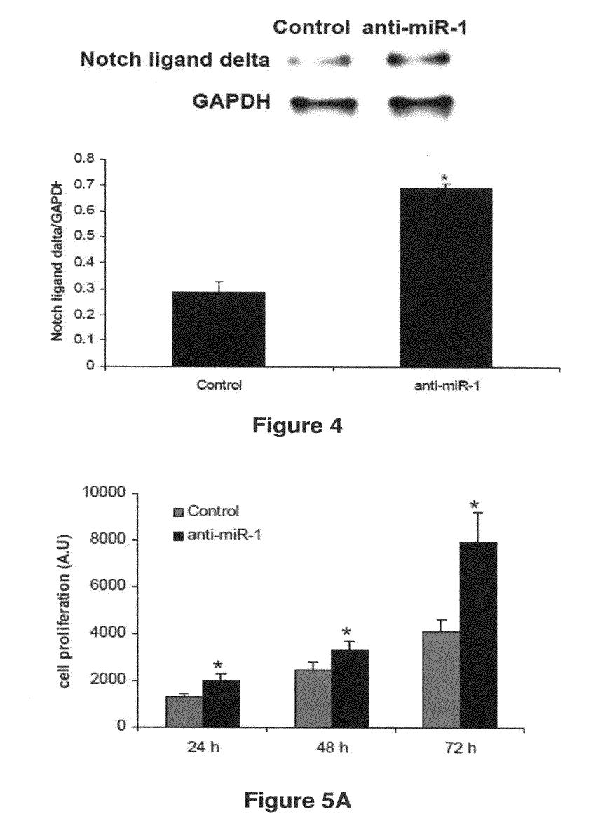

FIG. 4 shows that miR-1 targets Notch ligand delta expression and regulates its expression in human Keratinocytes. HaCaT (human keratinocyte) cells were transfected with anti-miR-1 or a non-targeting negative control. After 72 hours, protein was extracted. Western blot was run with 70 jig of protein. GAPHD was used as the housekeeping gene. p<0.05 compared to control.

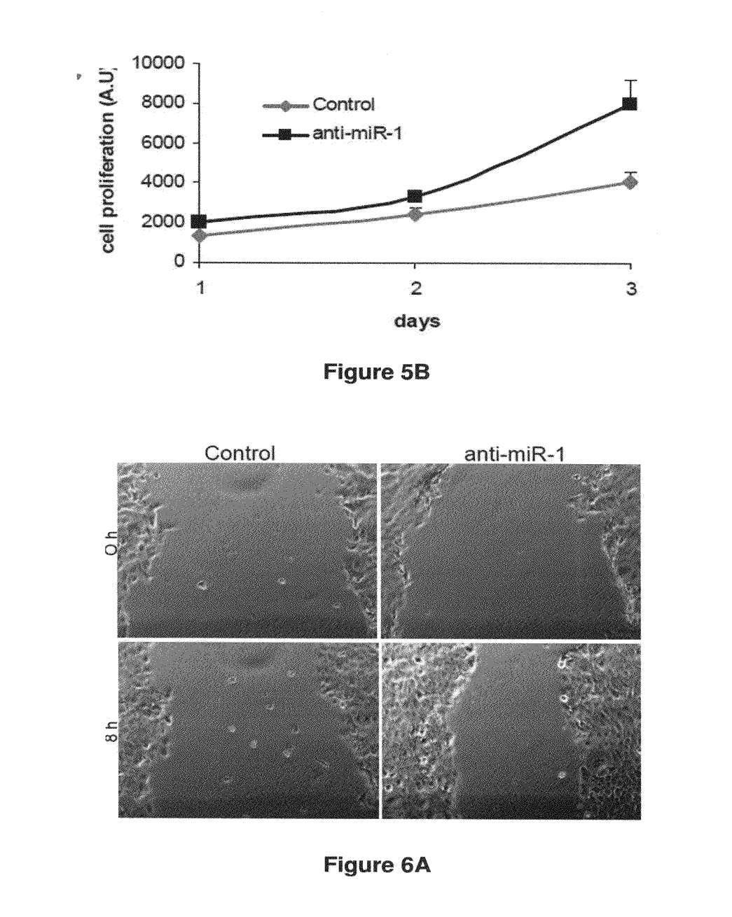

FIG. 5A (hours) and FIG. 5B (days) show that silencing miR-1 in human Keratinocytes induces an increase in cell proliferation. HaCaT (human keratinocytes) cells were transfected with anti-miR-1 or a non-targeting negative control. After 72 hours, cells were collected and reseeded for CY Quant proliferation assay. p<0.05 compared to control.

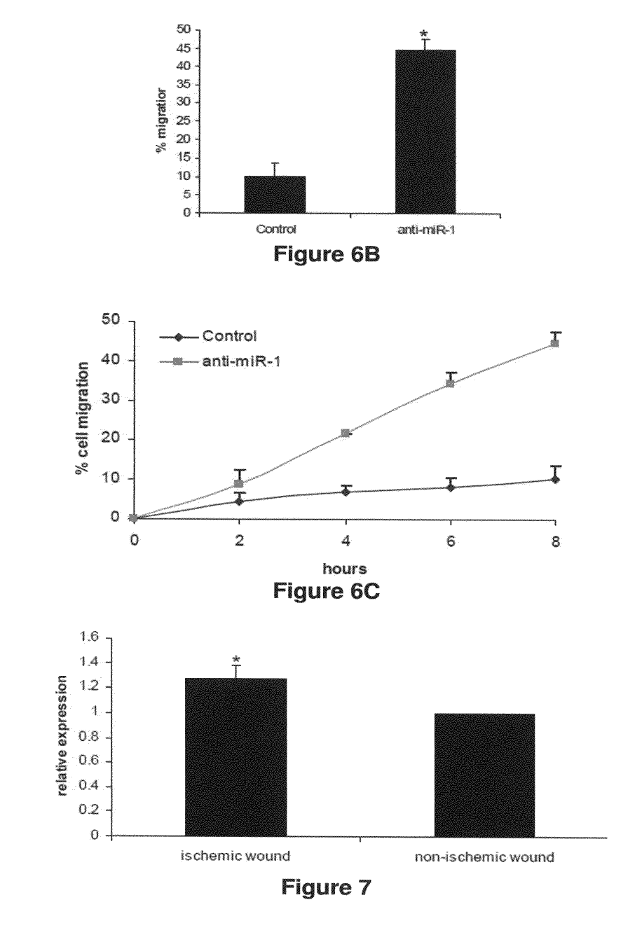

FIG. 6A, FIG. 6B and FIG. 6C show that silencing miR-1 in human Keratinocytes induces an increase in cell migration. HaCaT (human keratinocytes) cells were transfected with anti-miR-1 or a non-targeting negative control. After 72 hours, cells were collected and reseeded for migration assay. A scratch was performed and imaged every 2 h. 10× magnification. Percentage of closure was calculated. p<0.05 compared to control.

FIG. 7 shows that miR-1 down-regulation of expression is impaired in ischemic wounds. 3 mm punch wounds, Ischemic and non-ischemic were performed on the same animal using a flap technique. Wound samples were collected at day 7, miRNA were isolated, and Taqman® assays were performed. snoRNA202 was used as the housekeeping gene. N=3 paired. p<0.05 compared to control.

Examples of Therapeutic Methods

Methods for Altering Activity of MiRs

miR-directed therapies can be used to help in wound healing. By using miRNAs that change during the wound healing process, there is provided herein new targets for gene therapy. Such miRNAs target and regulate a range of genes that are connected to the specific phenotype. Therefore, such miRNA therapy increases the usefulness of gene therapy and can be useful to treat diseases.

Methods of the invention include modulating the activity of one or more miRNAs in a cell comprising introducing into a cell a miRNA modulator (which may be described generally herein as an miRNA); or inhibiting the activity of one or more miRNAs in a cell. The present invention also concerns inducing certain cellular characteristics by providing to a cell a particular nucleic acid, such as a specific synthetic miRNA molecule or a synthetic miRNA inhibitor molecule. However, in methods of the invention, the miRNA molecule or miRNA inhibitor need not be synthetic. They may have a sequence that is identical to a naturally occurring miRNA or they may not have any design modifications. In certain embodiments, the miRNA molecule and/or the miRNA inhibitor are synthetic, as discussed herein.

The particular nucleic acid molecule provided to the cell is understood to correspond to a particular miRNA in the cell, and thus, the miRNA in the cell is referred to as the “corresponding miRNA.” In situations in which a named miRNA molecule is introduced into a cell, the corresponding miRNA will be understood to be the induced or inhibited miRNA or induced or inhibited miRNA function. It is contemplated, however, that the miRNA molecule introduced into a cell may not necessarily be a mature miRNA but is capable of becoming or functioning as a mature miRNA under the appropriate physiological conditions.

The inventors believe that, in certain embodiments, a combination of miRNA may act at one or more points in cellular pathways of cells with aberrant phenotypes and that such combination may have increased efficacy on the target cell while not adversely effecting normal cells. Thus, a combination of miRNA may have a minimal adverse effect on a subject or subjects while supplying a sufficient therapeutic effect, such as amelioration of a condition, growth inhibition of a cell, death of a targeted cell, alteration of cell phenotype or physiology, slowing of cellular growth, sensitization to a second therapy, sensitization to a particular therapy, and the like.

Methods for Identifying Cells or Subjects

Methods include identifying a cell or subject in need of inducing those cellular characteristics. Also, it will be understood that an amount of a synthetic nucleic acid that is provided to a cell or organism is an “effective amount,” which refers to an amount needed (or a sufficient amount) to achieve a desired goal, such as inducing a particular cellular characteristic(s).

Certain embodiments of the methods include providing or introducing to a cell a nucleic acid molecule corresponding to a mature miRNA in the cell in an amount effective to achieve a desired physiological result.

Moreover, methods can involve providing synthetic or no synthetic miRNA molecules. It is contemplated that in these embodiments, that the methods may or may not be limited to providing only one or more synthetic miRNA molecules or only one or more no synthetic miRNA molecules. Thus, in certain embodiments, methods may involve providing both synthetic and no synthetic miRNA molecules. In this situation, a cell or cells are most likely provided a synthetic miRNA molecule corresponding to a particular miRNA and a no synthetic miRNA molecule corresponding to a different miRNA.

In some embodiments, there is a method for reducing or inhibiting cell proliferation comprising introducing into or providing to the cell an effective amount of (i) a miRNA molecule or (ii) a synthetic or nonsynthetic miRNA molecule that corresponds to a miRNA sequence. In certain embodiments the methods involve introducing into the cell an effective amount of (i) an miRNA molecule having a 5′ to 3′ sequence that is at least 90% complementary to the 5′ to 3′ sequence of one or more mature miRNA.

It will be understood in methods of the invention that a cell or other biological matter such as an organism (including subjects) can be provided a miRNA or miRNA molecule corresponding to a particular miRNA by administering to the cell or organism a nucleic acid molecule that functions as the corresponding miRNA once inside the cell. The form of the molecule provided to the cell may not be the form that acts as a miRNA once inside the cell.

In certain methods of the invention, there is a further step of administering the selected miRNA modulator to a cell, tissue, organ, or organism (collectively “biological matter”) in need of treatment related to modulation of the targeted miRNA or in need of the physiological or biological results discussed herein (such as with respect to a particular cellular pathway or result like decrease in cell viability).

Consequently, in some methods of the invention there is a step of identifying a subject in need of treatment that can be provided by the miRNA modulator(s). It is contemplated that an effective amount of a miRNA modulator can be administered in some embodiments. In particular embodiments, there is a therapeutic benefit conferred on the biological matter, where a “therapeutic benefit” refers to an improvement in the one or more conditions or symptoms associated with a disease or condition or an improvement in the prognosis, duration, or status with respect to the disease. It is contemplated that a therapeutic benefit includes, but is not limited to, a decrease in pain, a decrease in morbidity, or a decrease in a symptom.

Furthermore, it is contemplated that the miRNA compositions may be provided as part of a therapy to a subject, in conjunction with traditional therapies or preventative agents. Moreover, it is contemplated that any method discussed in the context of therapy may be applied as preventatively, particularly in a subject identified to be potentially in need of the therapy or at risk of the condition or disease for which a therapy is needed.

In addition, methods of the invention concern employing one or more nucleic acids corresponding to a miRNA and a therapeutic drug. The nucleic acid can enhance the effect or efficacy of the drug, reduce any side effects or toxicity, modify its bioavailability, and/or decrease the dosage or frequency needed.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

Citation of any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

1. A method of modulating gene expression in a skin cell comprising:
administering to the cell an amount of a therapeutic composition in an amount sufficient to down-regulate the expression of a miR-1 gene product in the skin cell.

2. (canceled)

3. The method of claim 1, wherein the therapeutic composition comprises one or more of: an anti-miR-1 gene product, an isolated miR-1 nucleic acid, a recombinant nucleic acid, an anti-miR-1 expression cassette, and a synthetic nucleic acid.

4. (canceled)

5. (canceled)

6. (canceled)

7. (canceled)

8. The method of claim 3, wherein the therapeutic composition comprises a double stranded nucleic acid molecule having one strand that is at least 95% complementary to at least a portion of a nucleic acid sequence encoding the anti-miR-1 gene product.

9. The method of claim 3, wherein the anti-miR-1 gene product inactivates expression of miR-1.

10. The method of claim 1, wherein the therapeutic composition further comprising a cell-penetrating peptide.

11. The method of claim 10, wherein the cell penetrating peptide is linked to the anti-miR-1 gene product.

12. The method of claim 3, wherein the anti-miR-1 gene product is administered by one or more of: topically to a wound, locally to a wound, included in a dressing that is applied topically to a wound, and locally to a wound via injection.

13. (canceled)

14. (canceled)

15. (canceled)

16. The method of claim 3, wherein the anti-miR-1 gene product is comprised in a pharmaceutical formulation.

17. The method of claim 16, wherein the pharmaceutical formulation is a lipid composition or a nanoparticle composition.

18. The method of claim 16, wherein the pharmaceutical formulation includes biocompatible and/or biodegradable molecules.

19. (canceled)

20. The method of claim 1, wherein the cell is in a subject having, suspected of having, or at risk of developing, a skin disease or condition.

21. The method of claim 20, wherein the condition associated with decreased vascularity wound healing disorders.

22. The method of claim 21, wherein the cell is a keratinocyte.

23. (canceled)

24. (canceled)

25. (canceled)

26. A method of promoting healing of a wound, comprising:
administering to at least one wound cell or tissue, an amount of a miR-1 inhibitory compound effective to promote healing of the wound.

27. (canceled)

28. The method of claim 26, wherein the wound is a chronic wound.

29. The method of claim 28, wherein the chronic wound is one or more of: a diabetic ulcer, a pressure ulcer, an arterial ulcer, a venous ulcer, an acute wound, or a surgical wound.

30. (canceled)

31. (canceled)

32. (canceled)

33. (canceled)

34. (canceled)

35. (canceled)

36. (canceled)

37. (canceled)

38. (canceled)

39. A composition useful for promoting healing of a wound comprising an amount of a miR-1 inhibitory compound effective to promote healing of wounded tissue.

40. (canceled)

41. (canceled)

42. (canceled)

43. (canceled)

44. The method of claim 1, wherein the cell is a mammalian cell.

45. The method of claim 44, wherein the mammalian cell is a human cell.
